(19) United States
(12) Patent Application Publication
(10) Pub. No.: US 2017/0333309 A1
(43) Pub. Date: Nov. 23, 2017

(54) DEODORIZING ACTIVE INGREDIENT COMBINATION

(71) Applicant: Henkel AG & Co. KGaA, Duesseldorf (DE)

(72) Inventor: Thomas Doering, Dormagen (DE)

(73) Assignee: Henkel AG & Co. KGaA, Duesseldorf (DE)

(21) Appl. No.: 15/593,089

(22) Filed: May 11, 2017

(30) Foreign Application Priority Data
May 18, 2016 (DE) 102016208526.3

(51) Int. Cl. A61K 8/26; A61K 8/37; A61K 8/58; A61Q 15/00

(52) U.S. Cl. CPC A61K 8/26; A61Q 15/00; A61K 8/37; A61K 8/585; A61K 2800/30

(57) ABSTRACT

A deodorizing cosmetic agent in the form of a suspension and related methods of use. The deodorizing cosmetic agent includes 1.0 to 35 wt % at least one hectorite, 55 to 95 wt % at least one oil, less than 3.0 wt % water. The deodorizing cosmetic agent does not include any aluminum-containing compounds.

### FIELD OF THE INVENTION

The present invention generally relates to cosmetic deodorant compositions having an improved effect, and methods using said compositions.

### BACKGROUND OF THE INVENTION

In many cultures, cleaning of and care for one's own body includes measures to counteract the emergence of excessive unpleasant-smelling body odor. The emergence of body odor is encouraged, inter alia, by perspiration, for which reason the cosmetic industry offers many body care agents that have been developed for use in areas of the body that have a high density of sweat glands, in particular, in the armpit area. These body care agents act as antiperspirants and/or deodorants.

Cosmetic antiperspirants of the prior art normally include at least one oil or fatty substance and odorant component or perfume and, in addition thereto, at least one antiperspirant salt. On one hand, the antiperspirant compounds used in these antiperspirants reduce the secretion of sweat from the body by temporarily narrowing and/or blocking the ducts of the sweat glands so that the amount of sweat can be reduced by about 20 to 60%. On the other hand, these antiperspirant compounds have an additional deodorizing effect due to the antimicrobial action thereof. Alkaline aluminum and aluminum-zirconium halides (which usually have been activated) are used as antiperspirant salts. It is also possible to use aluminum and aluminum-zirconium halides that have been stabilized with organic acids as complex ligands. People with sensitive skin may react with skin irritation to the antiperspirant salts.

Antiperspirants and deodorants may be prepared in the most varied forms of application, for example, as a powder, in the form of a stick, as an aerosol spray, liquid and gelatinous roll-on application, cream, gel, and as an impregnated flexible substrate (deodorant wipes).

Manufacturing the antiperspirants and deodorants in liquid form has advantages in relation to dosage and application, e.g., by means of an atomizer or spray. Low-viscous emulsion systems that are suitable for spraying, however, often do not have sufficient storage stability.

German patent application DE 2519860 A1 and European patent application EP 440387 A1 disclose antiperspirants and deodorants that include hydrophobically modified hectorite, in addition to other components. Corresponding hydrophobically modified hectorites have, however, proven inadequate in terms of the absorption of moisture and the reduction of odor.

In this context, the present invention addresses the industrial problem of providing a deodorant having high moisture absorption, high effectiveness against body odor, low residue formation, favorable sensory qualities and skin compatibility, and high storage stability and microbiological stability.

### BRIEF SUMMARY OF THE INVENTION

The cosmetic agents according to the present invention include at least one hectorite as the first essential component thereof. Three-layered clay minerals from the group consisting of smectites are called hectorites. The term “hectorite” therefore encompasses silicate minerals but not the hydrophobized derivatives thereof, such as are marketed under the name Bentone 38 (INCI: Quaternium-18 Hectorite) by the company Elementis.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now surprisingly been found that the aforementioned industrial problems are solved by aluminum-free compositions that include at least one hectorite. The present invention provides:

A deodorizing cosmetic agent in the form of a suspension, containing:
1.0 to 35 wt % at least one hectorite,
55 to 95 wt % at least one oil that is liquid at 20° C. and 1,013 hPa,
less than 3.0 wt % water
   wherein the deodorizing cosmetic agent does not include any aluminum-containing compounds.

The deodorizing cosmetic agent according to point 1, wherein the agent includes a hectorite having a degree of purity above 95%, preferably above 97% and, in particular, above 99%.

The deodorizing cosmetic agent according to any of the preceding points, wherein the agent includes the hectorite, relative to the total weight of the agent, in an amount of 2.0 to 20% and, in particular, 3.0 to 15 wt %.

The deodorizing cosmetic agent according to any of the preceding points, wherein at least one oil is selected from the group that has a vapor pressure of 0.01 kPa or more at 20° C.

The deodorizing cosmetic agent according to any of the preceding points, wherein at least one oil is selected from the group consisting of silicone oils, preferably from the group consisting of dialkyl and alkylaryl siloxanes, in particular, from the group consisting of cyclopentasiloxane, cyclohexasiloxane, dimethylpolysiloxane, and methylphenylpolysiloxane.

The deodorizing cosmetic agent according to any of the preceding points, wherein cyclopentasiloxane is included as an oil.

The deodorizing cosmetic agent according to any of the preceding points, wherein at least one oil is selected from the group consisting of esters of linear or branched saturated or unsaturated fatty acids, preferably from the group consisting of esters of linear or branched saturated or unsaturated C2-30 fatty acids with unsaturated C2-30 alkanols, in particular from the group consisting of hexyldecyl stearate, hexyldecyl laurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, and isotridecyl isononanoate.

The deodorizing cosmetic agent according to any of the preceding points, wherein 2-ethylhexyl palmitate is included as an oil.

The deodorizing cosmetic agent according to any of the preceding points, wherein 2-isopropyl myristate is included as an oil.

The deodorizing cosmetic agent according to any of the preceding points, wherein a mixture of dimethylsiloxane, cyclopentasiloxane, 2-ethylhexyl palmitate, and isopropyl myristate is included as the oil.

The deodorizing cosmetic agent according to any of the preceding points, wherein the agent includes, as the oil, a mixture of silicone oil and non-silicone oil and the weight ratio of silicone oil to non-silicone oil is 3:1 to 1:3, preferably 2:1 to 1:2 and, in particular, 3:2 to 2:3.

The deodorizing cosmetic agent according to any of the preceding points, wherein the agent includes oil, relative to the total weight of the agent, in amounts of 65 to 92% and, in particular, 80 to 92 wt %.

The deodorizing cosmetic agent according to any of the preceding points, wherein the agent includes water, relative to the total weight of the agent, in amounts below 2.0 wt % and, in particular, below 1.0 wt %.

The deodorizing cosmetic agent according to any of the preceding points, wherein the agent does not include any water.

The deodorizing cosmetic agent according to any of the preceding points, wherein the agent includes—relative to the total weight of the agent-0.5 to 6.0 wt %, preferably 1.0 to 5.0 wt % and, in particular, 2.0 to 4.0 wt % thickening agent.

The deodorizing cosmetic agent according to any of the preceding points, wherein the agent includes a thickening agent from the group consisting of hydrophobically modified silicates, preferably from the group consisting of hydrophobized hectorites, in particular, from the group consisting of hydrophobized hectorites with the INCI designation Quaternium-18 Hectorite.

The deodorizing cosmetic agent according to any of the preceding points, wherein the agent includes—relative to the total weight of the agent-1.0 to 10 wt %, preferably 2.0 to 8.0 wt % and, in particular, 3.0 to 6.0 wt % at least one enzyme inhibitor.

The deodorizing cosmetic agent according to any of the preceding points, wherein the agent includes an enzyme inhibitor from the group consisting of trialkyl citric acid esters, preferably, triethyl citrate or zinc glycinate, in particular, triethyl citrate.

The deodorizing cosmetic agent according to any of the preceding points, wherein the agent is composed of hectorite, oil, and enzyme inhibitor to at least 80 wt %, preferably at least 85 wt % and, in particular, at least 90 wt %, relative to the total weight of the agent.

An aerosol delivery device containing propellant gas and the deodorizing cosmetic agent according to any of the preceding points.

A pump sprayer containing the deodorizing cosmetic agent according to any of the preceding points.

Use of the deodorizing agent according to any of the preceding points to prevent and/or reduce body odor.

A non-therapeutic cosmetic method for preventing and/or reducing body odor, in which the cosmetic agent according to any of the preceding points is applied to the skin, in particular, to the skin of the armpits, and left on the skin for at least 1 hour, preferably for at least 2 hours, preferably for at least 4 hours, in particular, for at least 6 hours.

The use of hectorites with a specific degree of purity has proven advantageous for the industrial effect, in particular, the absorption of odor, the formation of residue on textiles, and the sensory qualities. The term “degree of purity” refers then to the absence of mineral impurities. Deodorizing cosmetic agents that include a hectorite having a degree of purity above 95%, preferably above 97% and, in particular, above 99% are preferred according to the present invention.

The proportion by weight of the hectorite to the total weight of the deodorizing cosmetic agent is preferably 2.0 to 20 wt % and, in particular, 3.0 to 15 wt %.

A second essential component of the agents according to the present invention is the oil that is liquid at 20° C. and 1,013 hPa.

The term “cosmetic oil” is understood to mean an oil that is suitable for cosmetic use and is not miscible with water. Moreover, the cosmetic oil used according to the present invention involves neither odorants nor essential oils.

According to a preferred embodiment, the cosmetic oil that is liquid at 20° C. and 1,013 hPa is selected from the group consisting of:
(i) volatile cyclic silicone oils, in particular cyclotrisiloxane, cyclotetrasiloxane, cyclopentasiloxane, and cyclohexasiloxane, and linear silicone oils having 2 to 10 siloxane units, in particular hexamethyldisiloxane, octamethyltrisiloxane, and decamethyltetrasiloxane;
(ii) volatile non-silicone oils, in particular liquid paraffin oils and isoparaffin oils, such as isodecane, isoundecane, isododecane, isotridecane, isotetradecane, isopentadecane, isohexadecane, and isoeicosane;
(iii) non-volatile silicone oils, in particular higher molecular linear polyalkylsiloxanes;
(iv) non-volatile non-silicone oils, in particular the esters of linear or branched, saturated or unsaturated C₂₋₃₀ fatty alcohols with linear or branched, saturated or unsaturated C₂₋₃₀ fatty acids, which may be hydroxylated, the C_{8-C22} fatty alcohol esters of monohydric or polyhydric C₂-C₇ hydroxycarboxylic acids, the triethyl citrates, the branched saturated or unsaturated C₆₋₃₀ fatty alcohols, the mono-, di-, and triglycerides of linear or branched, saturated or unsaturated, optionally hydroxylated C₈₋₃₀ fatty acids, the dicarboxylic acid esters of linear or branched C₂-C₁₀ alkanols, the addition products of ethylene oxide and/or propylene oxide with monohydric or polyhydric C₃₋₂₂ alkanols, which may optionally be esterified, the symmetrical, asymmetrical, or cyclic esters of carbonic acid with fatty alcohols, the esters of dimers of unsaturated C₁₂₋₂₂ fatty acids with monohydric linear, branched, and cyclic C₂₋₁₈ alkanols or C₂₋₆ alkanols, the benzoic acid esters of linear or branched C₈₋₂₂ alkanols, such as benzoic acid C₁₂₋₁₅ alkyl esters and benzoic acid isostearyl ester and benzoic acid octyldodecyl ester, the synthetic hydrocarbons such as polyisobutene and polydecene, the alicyclic hydrocarbons; and
(v) and mixtures thereof

The term “volatile cosmetic oil” refers to cosmetic oils that—20° C. and an ambient pressure of 1,013 hPa—have a vapor pressure of 2.66 Pa to 40,000 Pa (0.02 to 300 mmHg), preferably 10 to 12,000 Pa (0.1 to 90 mmHg), further preferably 13 to 3,000 Pa (0.1 to 23 mmHg) and, in particular, 15 to 500 Pa (0.1 to 4 mmHg).

The term “non-volatile cosmetic oils” is understood to mean cosmetic oils that have a vapor pressure of less than 2.66 Pa (0.02 mmHg) at 20° C. and an ambient pressure of 1,013 hPa.

It is especially preferred to use volatile cosmetic oils, in particular, those cosmetic oils that have a vapor pressure of 0.01 kPa or more at 20° C. Industrially especially advantageous oils are those from the group consisting of silicone oils, preferably from the group consisting of dialkyl and alkylaryl siloxanes, wherein cyclopentasiloxane, cyclohexasiloxane, dimethylpolysiloxane, and methylphenylpolysiloxane are especially preferred. Cyclopentasiloxane is very especially preferred.

Alternatively or in addition to the aforementioned silicone oils, cosmetic agents include at least one oil selected from the group consisting of the esters of linear or branched saturated or unsaturated fatty acids. A preferred group is the group consisting of esters of linear or branched saturated or unsaturated C₂-30 fatty acids with unsaturated C₂-30 alkanols, in particular the group consisting of hexyldecyl stearate, hexyldecyl laurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, and isotridecyl isononanoate. It is very especially preferred to use 2-ethylhexyl palmitate and isopropyl myristate.

If mixtures of silicone oil and non-silicone oil are used in the deodorizing cosmetic agents, then the weight ratio of silicone oil to non-silicone oil is preferably 3:1 to 1:3, preferably 2:1 to 1:2 and, in particular, 3:2 to 2:3.

The of mixtures of oils has proven especially advantageous for the absorption of moisture and odor, but in particular with respect to the formation of residue on textiles and the sensory properties of the deodorizing cosmetic agents. An especially advantageous oil mixture comprises dimethylsiloxane, cyclopentasiloxane, 2-ethylhexyl palmitate, and isopropyl myristate.

Preferred deodorizing cosmetic agents include, relative to the total weight thereof, oil in amounts of 65 to 92 wt % and, in particular, 80 to 92 wt %.

In order to ensure sufficient moisture absorption, the deodorizing cosmetic agents preferably include less than 2.0 wt % and, in particular, less than 1.0 wt % water relative to the total weight thereof. Deodorizing cosmetic agents that do not include any water are especially preferable.

The cosmetic agents do not include any aluminum-containing compounds. Within the scope of the present invention, the term “aluminum-containing compounds” is understood to mean antiperspirant aluminum salts and aluminum zirconium salts.

To adjust the viscosity, the cosmetic agents may include thickening agents as other suitable additives. The thickening agent is different from the components hectorite and oil that are compulsorily included in the cosmetic agents. Especially preferred thickening agents are the hydrophobically modified silicates, in particular, the hydrophobized hectorites, especially preferable, the hydrophobized hectorites with the INCI designation Quaternium-18 Hectorite, such as have been marketed under the tradename Benton 38 (Elementis). As previously mentioned, these thickening agents, in particular, the hydrophobically modified hectorites are not identical to the hectorites that are compulsorily included in the deodorizing cosmetic agents.

Preferred cosmetic agents include the thickening agent, in particular, the hydrophobically modified silicates, especially preferably, the hydrophobized hectorite in amounts of 0.5 to 6.0 wt %, preferably 1.0 to 5.0 wt % and, in particular, 2.0 to 4.0 wt %, in each case relative to the total weight of the cosmetic agent.

Very especially preferred cosmetic agents include a hydrophobized hectorite with the INCI designation Quaternium-18 Hectorite in amounts of 0.5 to 6.0 wt %, preferably 1.0 to 5.0 wt % and, in particular, 2.0 to 4.0 wt %, relative to the total weight thereof

Cosmetic deodorants counteract, mask, or eliminate body odors. Body odors are produced by the action of skin bacteria on apocrine sweat, wherein unpleasant-smelling degradation products are formed. Correspondingly, preferred deodorants include active ingredients that act as germ inhibitors, enzyme inhibitors, odor absorbers, or odor maskers.

Essentially all substances that act against gram-positive bacteria are suitable as germ inhibitors that may optionally be added to the cosmetic agents according to the present invention. Suitable examples include 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3, 5 -dimethylphenol, 2,2 ‘-methylene-bis-(6-bromo-4-chlorophenol), 3-methyl-4-(1-methylethyl)-phenol, 2-benzyl-4-chlorophenol, 3 -(4-chlorophenoxy)-propane-1,2-diol, 3 -iodo-2-propinyl butyl carbamate, chlorhexidine, 3,4,4′-trichlorocarbanilide (TTC), antibacterial odorants, thymol, thyme oil, eugenol, clove oil, menthol, mint oil, farnesol, phenoxyethanol, glycerol monolaurate (GML), diglycerol monocaprate (DMC), salicylic acid-N-alkylamides such as, for example, salicylic acid-n-octyl amide or salicylic acid-n-decyl amide.

Enzyme inhibitors may be added to the cosmetic agents according to the present invention, provided that they do not impair the enzyme activities that bring about the biocide activation or release in the presence of micro-organisms to be controlled. In accordance with this condition, for example, esterase inhibitors may possibly be suitable enzyme inhibitors. This preferably entails trialkyl citrates such as trimethyl citrate, tripropyl citrate, triisopropyl citrate, tributyl citrate, and, in particular, triethyl citrate (Hydagen® CAT, Henkel KGaA, Dusseldorf/FRG). The substances inhibit the enzyme activity and reduce thereby the formation of odor. Other substances that may be considered as esterase inhibitors are sterol sulfates or phosphates such as, for example, lanosterol, cholesterol, campesterol, stigmasterol and cytosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Trialkyl citric acid esters have proven to be especially effective enzyme inhibitors, in combination with the active ingredient combination according to the present invention. Use thereof, in particular, use of triethyl citrate and zinc glycinate, had an unexpectedly advantageous effect on the odor formation of the cosmetic agents. Deodorizing cosmetic agents that include a trialkyl citric acid ester, preferably triethyl citrate or zinc glycinate, in particular, triethyl citrate, as an enzyme inhibitor are therefore preferred.

The proportion by weight of the enzyme inhibitor to the total weight of the cosmetic agent is preferably 1.0 to 10 wt %, preferably 2.0 to 8.0 wt % and, in particular, 3.0 to 6.0 wt %.

Cosmetic agents composed largely of the ingredients hectorite, oil, and enzyme inhibitor have proven especially effective. Due to the cosmetic effect and with the aim of minimizing the complexity of the preparation of these agents, it is therefore preferable that the cosmetic agent be composed of hectorite, oil, and enzyme inhibitor to at least 80 wt %, preferably at least 85 wt % and, in particular, at least 90 wt % relative to the total weight thereof.

In addition to the aforementioned ingredients, the deodorizing cosmetic agents may include additional active ingredients and auxiliaries. The group of these further active ingredients and auxiliaries includes, for example, the odorants.

Especially preferred cosmetic agents according to the present invention further include at least one odorant. The definition of an odorant within the meaning of the present invention coincides with the definition that would be known to a person skilled in the art, such as can be found in the RÖMPP Chemie Lexikon, as of December 2007. An odorant, then, is a chemical compound having odor and/or taste, which excites the receptors of the hair cells of the olfactory system (adequate stimulus). The physical and chemical properties necessary for this are a low molar mass of at most 300 g/mol, a high vapor pressure, minimal water solubility and high lipid solubility, as well as weak polarity and the presence of at least one osmophoric group in the molecule In order to distinguish volatile low-molecular-weight substances that are usually (and also for purposes of the present invention) viewed and utilized not as fragrances but instead principally as solvents, for example ethanol, propanol, isopropanol, and acetone, from odorants, the odorants according to the present invention have a molar mass of 74 to 300 g/mol, include at least one osmophoric group in the molecule, and have an odor and/or taste, i.e. they excite the receptors of the hair cells of the olfactory system.

Perfumes, perfume oils, or perfume oil components may be used as odorants. Perfume oils or fragrances may, according to the present invention, be individual odorant compounds, such as synthetic products of the ester, ether, aldehyde, ketone, alcohol, and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate, and jasmecyclate. The ethers include, for example, benzyl ethyl ether and ambroxan; the aldehydes, for example, the linear alkanals having 8 to 18 carbon atoms, citral, citronellal, citronellyloxyacetaldehyde, cyclamenaldehyde, lilial, and bourgeonal; the ketones, for example, the ionones, a-isomethylionone, and methyl cedryl ketone; the alcohols, anethole, citronellol, eugenol, geraniol, linalool, phenylethyl alcohol, and terpineol; and the hydrocarbons include principally the terpenes such as limonene and pinene. It is, however, preferable to use mixtures of different odorants which together produce an appealing odorous note. Preferred cosmetic agents according to the present invention include at least one fragrance component in a total amount of 0.00001 to 10 wt %, preferably 0.5 to 5 wt %, extremely preferably 1 to 4 wt %, in each case relative to the total weight of the agent.

The compositions of some preferred cosmetic agents can be found in the following tables (all amounts given in wt % are relative to the total weight of the cosmetic agent, unless otherwise indicated).

| | Formula 1 | Formula 2 | Formula 3 | Formula 4 | Formula 5 |
|---|---|---|---|---|---|
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa

| | | | | | Formula |
|---|---|---|---|---|---|
| | Formula 6 | Formula 7 | Formula 8 | Formula 9 | 10 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least one oil selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 11 | 12 | 13 | 14 | 15 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 16 | 17 | 18 | 19 | 20 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 21 | 22 | 23 | 24 | 25 |
| Hectorite | 1.0 to 35 | 2.0 to 20 | 2.0 to 20 | 3.0 to 15 | 3.0 to 15 |
| Oil* | 55 to 95 | 55 to 95 | 65 to 95 | 65 to 95 | 80 to 95 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
the oils dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 26 | 27 | 28 | 29 | 30 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa

| | Formula | Formula | Formula | Formula | Formula |
|---|---|---|---|---|---|
| | 31 | 32 | 33 | 34 | 35 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa

| | Formula | Formula | Formula | Formula | Formula |
|---|---|---|---|---|---|
| | 36 | 37 | 38 | 39 | 40 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least one oil selected from the group consisting of
dimethylpolysiloxane, cyclopentasiloxane, 2-ethylhexyl palmitate, and
isopropyl myristate.

| | Formula | Formula | Formula | Formula | Formula |
|---|---|---|---|---|---|
| | 41 | 42 | 43 | 44 | 45 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 46 | 47 | 48 | 49 | 50 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate, and
isopropyl myristate.

| | Formula | Formula | Formula | Formula | Formula |
|---|---|---|---|---|---|
| | 51 | 52 | 53 | 54 | 55 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
the oils dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 56 | 57 | 58 | 59 | 60 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least one oil selected from the group consisting of dimethylpolysiloxane,
cyclopentasiloxane, 2-ethylhexyl palmitate, and isopropyl myristate.

| | Formula | Formula | Formula | Formula | Formula |
|---|---|---|---|---|---|
| | 61 | 62 | 63 | 64 | 65 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 66 | 67 | 68 | 69 | 70 |
| Hectorite | 1.0 to 35 | 2.0 to 20 | 2.0 to 20 | 3.0 to 15 | 3.0 to 15 |
| Oil* | 55 to 95 | 55 to 95 | 65 to 95 | 65 to 95 | 80 to 95 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 71 | 72 | 73 | 74 | 75 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
the oils dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 76 | 77 | 78 | 79 | 80 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa
**Hectorite having a degree of purity above 95%, preferably above 97%
and, in particular, above 99%.

| | Formula | Formula | Formula | Formula | Formula |
|---|---|---|---|---|---|
| | 81 | 82 | 83 | 84 | 85 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises at
least one oil selected from the group consisting of dimethylpolysiloxane,
cyclopentasiloxane, 2-ethylhexyl palmitate, and isopropyl myristate.
** Hectorite having a degree of purity above 95%, preferably above
97% and, in particular, above 99%.

| | Formula | Formula | Formula | Formula | Formula |
|---|---|---|---|---|---|
| | 86 | 87 | 88 | 89 | 90 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises at
least two oils selected from the group consisting of dimethylpolysiloxane,
cyclopentasiloxane, 2-ethylhexyl palmitate, and isopropyl myristate.
**Hectorite having a degree of purity above 95%, preferably above 97%
and, in particular, above 99%.

| | Formula | Formula | Formula | Formula | Formula |
|---|---|---|---|---|---|
| | 91 | 92 | 93 | 94 | 95 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate, and
isopropyl myristate.
**Hectorite having a degree of purity above 95%, preferably above 97%
and, in particular, above 99%.

| | Formula | Formula | Formula | Formula | Formula |
|---|---|---|---|---|---|
| | 96 | 97 | 98 | 99 | 100 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
the oils dimethylpolysiloxane, cyclopentasiloxane, 2-ethylhexyl palmitate,
and isopropyl myristate.
**Hectorite having a degree of purity above 95%, preferably above 97%
and, in particular, above 99%.

| | Formula | Formula | Formula | Formula | Formula |
|---|---|---|---|---|---|
| | 101 | 102 | 103 | 104 | 105 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa
**Hectorite having a degree of purity above 95%, preferably above 97%
and, in particular, above 99%.

| | Formula | Formula | Formula | Formula | Formula |
|---|---|---|---|---|---|
| | 106 | 107 | 108 | 109 | 110 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa
**Hectorite having a degree of purity above 95%, preferably above
97% and, in particular, above 99%.

| | Formula | Formula | Formula | Formula | Formula |
|---|---|---|---|---|---|
| | 111 | 112 | 113 | 114 | 115 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises at
least one oil selected from the group consisting of dimethylpolysiloxane,
cyclopentasiloxane, 2-ethylhexyl palmitate, and isopropyl myristate.
**Hectorite having a degree of purity above 95%, preferably above 97%
and, in particular, above 99%.

| | Formula | Formula | Formula | Formula | Formula |
|---|---|---|---|---|---|
| | 116 | 117 | 118 | 119 | 120 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises at
least two oils selected from the group consisting of dimethylpolysiloxane,
cyclopentasiloxane, 2-ethylhexyl palmitate, and isopropyl myristate.
**Hectorite having a degree of purity above 95%, preferably above 97%
and, in particular, above 99%.

| | Formula | Formula | Formula | Formula | Formula |
|---|---|---|---|---|---|
| | 121 | 122 | 123 | 124 | 125 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises at
least three oils selected from the group consisting of dimethylpolysiloxane,
cyclopentasiloxane, 2-ethylhexyl palmitate, and isopropyl myristate.
**Hectorite having a degree of purity above 95%, preferably above
97% and, in particular, above 99%.

| | Formula | Formula | Formula | Formula | Formula |
|---|---|---|---|---|---|
| | 126 | 127 | 128 | 129 | 130 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
the oils dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 131 | 132 | 133 | 134 | 135 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises at
least one oil selected from the group consisting of
dimethylpolysiloxane, cyclopentasiloxane, 2-ethylhexyl palmitate, and
isopropyl myristate.
**Hectorite having a degree of purity above 95%, preferably above 97%
and, in particular, above 99%.

| | Formula | Formula | Formula | Formula | Formula |
|---|---|---|---|---|---|
| | 136 | 137 | 138 | 139 | 140 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate, and
isopropyl myristate.
**Hectorite having a degree of purity above 95%, preferably above 97%
and, in particular, above 99%.

| | Formula | Formula | Formula | Formula | Formula |
|---|---|---|---|---|---|
| | 141 | 142 | 143 | 144 | 145 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate, and
isopropyl myristate.
**Hectorite having a degree of purity above 95%, preferably above 97%
and, in particular, above 99%.

| | Formula | Formula | Formula | Formula | Formula |
|---|---|---|---|---|---|
| | 146 | 147 | 148 | 149 | 150 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
the oils dimethylpolysiloxane, cyclopentasiloxane, 2-ethylhexyl palmitate,
and isopropyl myristate.
**Hectorite having a degree of purity above 95%, preferably above 97%
and, in particular, above 99%.

The compositions of some preferred thickened cosmetic agents can be found in the following tables (all amounts given in wt % are relative to the total weight of the cosmetic agent, unless otherwise indicated). As thickening agents, it is preferred to use hydrophobized hectorites, in particular, hydrophobized hectorites with the INCI designation Quaternium-18 Hectorite.

| | Formula | Formula | Formula | Formula | Formula |
|---|---|---|---|---|---|
| | 151 | 152 | 153 | 154 | 155 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa

| | Formula | Formula | Formula | Formula | Formula |
|---|---|---|---|---|---|
| | 156 | 157 | 158 | 159 | 160 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least one oil selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 161 | 162 | 163 | 164 | 165 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate, and
isopropyl myristate.

| | Formula | Formula | Formula | Formula | Formula |
|---|---|---|---|---|---|
| | 166 | 167 | 168 | 169 | 170 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 171 | 172 | 173 | 174 | 175 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil
comprises the oils dimethylpolysiloxane, cyclopentasiloxane,
2-ethylhexyl palmitate, and isopropyl myristate.

| | Formula | Formula | Formula | Formula | Formula |
|---|---|---|---|---|---|
| | 176 | 177 | 178 | 179 | 180 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa

| | Formula | Formula | Formula | Formula | Formula |
|---|---|---|---|---|---|
| | 181 | 182 | 183 | 184 | 185 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa

| | Formula | Formula | Formula | Formula | Formula |
|---|---|---|---|---|---|
| | 186 | 187 | 188 | 189 | 190 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least one oil selected from the group consisting of
dimethylpolysiloxane, cyclopentasiloxane, 2-ethylhexyl palmitate, and
isopropyl myristate.

| | Formula | Formula | Formula | Formula | Formula |
|---|---|---|---|---|---|
| | 191 | 192 | 193 | 194 | 195 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 196 | 197 | 198 | 199 | 200 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 201 | 202 | 203 | 204 | 205 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil
comprises the oils dimethylpolysiloxane, cyclopentasiloxane,
2-ethylhexyl palmitate, and isopropyl myristate.

| | Formula | Formula | Formula | Formula | Formula |
|---|---|---|---|---|---|
| | 206 | 207 | 208 | 209 | 210 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least one oil selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 211 | 212 | 213 | 214 | 215 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 216 | 217 | 218 | 219 | 220 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil
comprises at least three oils selected from the group consisting
of dimethylpolysiloxane, cyclopentasiloxane, 2-ethylhexyl palmitate,
and isopropyl myristate.

| | Formula | Formula | Formula | Formula | Formula |
|---|---|---|---|---|---|
| | 221 | 222 | 223 | 224 | 225 |
| Hectorite | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil
comprises the oils dimethylpolysiloxane, cyclopentasiloxane,
2-ethylhexyl palmitate, and isopropyl myristate.

| | Formula | Formula | Formula | Formula | Formula |
|---|---|---|---|---|---|
| | 226 | 227 | 228 | 229 | 230 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa
**Hectorite having a degree of purity above 95%, preferably above
97% and, in particular, above 99%.

| | Formula | Formula | Formula | Formula | Formula |
|---|---|---|---|---|---|
| | 231 | 232 | 233 | 234 | 235 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least one oil selected from the group consisting of
dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 236 | 237 | 238 | 239 | 240 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 241 | 242 | 243 | 244 | 245 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 246 | 247 | 248 | 249 | 250 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil
comprises the oils dimethylpolysiloxane, cyclopentasiloxane,
2-ethylhexyl palmitate, and isopropyl myristate.
**Hectorite having a degree of purity above 95%, preferably above
97% and, in particular, above 99%.

| | Formula | Formula | Formula | Formula | Formula |
|---|---|---|---|---|---|
| | 251 | 252 | 253 | 254 | 255 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa
**Hectorite having a degree of purity above 95%, preferably above
97% and, in particular, above 99%.

| | Formula | Formula | Formula | Formula | Formula |
|---|---|---|---|---|---|
| | 256 | 257 | 258 | 259 | 260 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa
**Hectorite having a degree of purity above 95%, preferably above
97% and, in particular, above 99%.

| | Formula | Formula | Formula | Formula | Formula |
|---|---|---|---|---|---|
| | 261 | 262 | 263 | 264 | 265 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least one oil selected from the group consisting of
dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 266 | 267 | 268 | 269 | 270 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 271 | 272 | 273 | 274 | 275 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 276 | 277 | 278 | 279 | 280 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Enzyme | 1.0 to 10 | 1.0 to 10 | 2.0 to 8.0 | 2.0 to 8.0 | 3.0 to 6.0 |
| inhibitor | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil
comprises the oils dimethylpolysiloxane, cyclopentasiloxane,
2-ethylhexyl palmitate, and isopropyl myristate.
**Hectorite having a degree of purity above 95%, preferably above
97% and, in particular, above 99%.

| | Formula | Formula | Formula | Formula | Formula |
|---|---|---|---|---|---|
| | 281 | 282 | 283 | 284 | 285 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least one oil selected from the group consisting of
dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 286 | 287 | 288 | 289 | 290 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least two oils selected from the group consisting of
dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 291 | 292 | 293 | 294 | 295 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil comprises
at least three oils selected from the group consisting of
dimethylpolysiloxane, cyclopentasiloxane, 2-ethylhexyl palmitate,
and isopropyl myristate.
**Hectorite having a degree of purity above 95%, preferably above
97% and, in particular, above 99%.

| | Formula | Formula | Formula | Formula | Formula |
|---|---|---|---|---|---|
| | 296 | 297 | 298 | 299 | 300 |
| Hectorite** | 1.0 to 35 | 2.0 to 20 | 2.0 to 20 | 3.0 to 15 | 3.0 to 15 |
| Oil* | 55 to 95 | 55 to 92 | 65 to 92 | 65 to 92 | 80 to 92 |
| Triethyl | 1.0 to 10 | 1.0 to 10 | 2.0 to 8.0 | 2.0 to 8.0 | 3.0 to 6.0 |
| citrate | | | | | |
| Thickening | 0.5 to 6.0 | 1.0 to 5.0 | 1.0 to 5.0 | 1.0 to 5.0 | 2.0 to 4.0 |
| agent | | | | | |
| Water | <3.0 | <2.0 | <2.0 | <1.0 | <1.0 |
| optional | up to 100 | up to 100 | up to 100 | up to 100 | up to 100 |
| additives | | | | | |

*oil that is liquid at 20° C. and 1,013 hPa, wherein the oil
comprises the oils dimethylpolysiloxane, cyclopentasiloxane,
2-ethylhexyl palmitate, and isopropyl myristate.
**Hectorite having a degree of purity above 95%, preferably above
97% and, in particular, above 99%.

The deodorizing cosmetic agent may be applied by means of different methods. According to a preferred embodiment, the antiperspirant cosmetic agent is prepared as a spray application. The spray application is performed with a spray device containing a container filled with the deodorizing cosmetic agent. The filling may be under the pressure of a propellant (pressure gas can, pressure gas packaging, aerosol packaging), or may entail a pump sprayer that is used mechanically, without propellant gas (pump spray/squeeze bottle). A preferred claimed subject matter is therefore an aerosol delivery device containing propellant gas and a deodorizing cosmetic agent according to the present invention. When propellant gas is forgone, the resulting propellant-free deodorizing agents are preferably applied by means of a pump spray.

The deodorizing agent may thus furthermore be manufactured as a roll-on. The application may take place with, for example a roller ball applicator. Such rollers have a ball bearing, mounted within a ball bed, which can be moved by motion over a surface. Then, the ball takes up some of the antiperspirant cosmetic agent according to the present invention to be distributed and transports same to the surface to be treated.

A suitable alternative form of application of the deodorizing cosmetic agent is combination with a disposable substrate, e.g., a substrate selected from the group consisting of cloths, pads, and pledgets. Particularly preferred are moist wipes, i.e., moist wipes which are prepackaged, preferably individually packaged, for the user, such as are well known, for example, from the area of glass cleaning or from the area of moist toilet wipes. Such moist wipes, which advantageously may also include preservatives, are impregnated or loaded with a deodorizing cosmetic agent according to the present invention and preferably individually packaged. They may be used, for example, as a deodorant wipe, which is of particular interest for usage while out and about. Preferred substrate materials are selected from porous flat wipes. They may be composed of a fibrous or cellular flexible material that has sufficient mechanical stability and, at the same time, softness for use on skin. These wipes include wipes made of woven and nonwoven (fleece) synthetic and natural fibers, felt, paper, or foam, such as hydrophilic polyurethane foam. According to the present invention, preferred deodorizing or antiperspirant substrates may be obtained by soaking or impregnating, or also melting, an antiperspirant cosmetic agent according to the present invention on a substrate.

Another subject matter being claimed is a non-therapeutic cosmetic method for preventing and/or reducing body odor, in which the cosmetic agent according to the present invention is applied to the skin, in particular, to the skin of the armpits, and left on the skin for at least 1 hour, preferably for at least 2 hours, preferably for at least 4 hours, in particular, for at least 6 hours.

As previously mentioned, the cosmetic agents according to the present invention have an advantageous effect on the formation of body odor. The use of these cosmetic agents to prevent and/or reduce body odor is another subject matter of the present invention.

### EXAMPLES

The following sprayable deodorant suspensions were produced (amounts given in wt %).

| | 1 | 2 | 3 | 4 | 5 | 6 | 7 | 8 |
|---|---|---|---|---|---|---|---|---|
| Cyclo- | 31.6 | 36.6 | 26.6 | 31.6 | 35.6 | 41.6 | 31.6 | 31.6 |
| pentasiloxane | | | | | | | | |
| Dimethicone | 10.0 | 10.0 | 10.0 | 10.0 | 10.0 | 10.0 | 10.0 | 10.0 |
| (5 cSt) | | | | | | | | |
| 2-Ethylhexyl | 30.0 | 30.0 | 30.0 | 30.0 | 30.0 | 30.0 | 30.0 | 30.0 |
| palmitate | | | | | | | | |
| Isopropyl | 6.0 | 6.0 | 6.0 | 6.0 | 6.0 | 6.0 | 6.0 | 6.0 |
| myristate | | | | | | | | |
| Triethyl citrate | 4.0 | 4.0 | 4.0 | 4.0 | 4.0 | 4.0 | 4.0 | 4.0 |
| Bentone | 2.5 | 2.5 | 2.5 | 2.5 | 2.5 | 2.5 | 2.5 | 12.5 |
| 38 V CG | | | | | | | | |
| Bentone MA | — | — | — | 5.0 | 3.0 | — | 10.0 | — |
| Bentone EW | 10.0 | 5.0 | 15.0 | 5.0 | 3.0 | — | — | — |
| Propylene | 0.9 | 0.9 | 0.9 | 0.9 | 0.9 | 0.9 | 0.9 | 0.9 |
| carbonate | | | | | | | | |
| Perfume | 5.0 | 5.0 | 5.0 | 5.0 | 5.0 | 5.0 | 5.0 | 5.0 |

Aerosol cans were filled with the deodorants at a weight ratio of 1:4 with the propellant propane/butane (15/85).

In subsequent testing, the deodorants containing Bentone MA and Bentone EW proved to be considerably superior to the other formulations with respect to moisture absorption and odor absorption.

To determine the odor absorption ability, sweat solutions with characteristic body odors were assessed in terms of the odor intensity. First, different types of hectorite were suspended in silicone oil (Dow Corning 2-1184) and then 0.3 mL thereof was applied to a filter paper (Filter Black Ribbon; MN 640 W no. 41; d=55 mm). After 1 hour of storage at room temperature, the filter papers were transferred to an artificial sweat solution and placed in a 1,000-mL tube with a screw cap. The artificial sweat solution is composed of characteristic volatile short-chain fatty acids (including 3-methyl-3-hydroxy-hexane acid). The glasses were stored for 24 hours at 37° C. The odor intensity was assessed by a trained panel (n=10).

To measure the moisture absorption of the hectorite types, 10 g of the pure substances was placed in a Petri dish and dried for 16 hours at 30*C. Then, the filled Petri dishes were placed for 24 hours in a desiccator at 30° C. and 92% relative humidity. The absorbed moisture is detected by determination of the weight.

The Bentone EW-based formulations have advantages over the Bentone MA-based formulations with respect to residue formation and sensory properties.

To assess the sensory properties, the formulations were sprayed onto the inside of the forearms. The assessment was carried out by trained experts 1 minute after application (1=dry, 5=sticky). The white residue was assessed immediately after spraying onto black cardboard, by comparison with a reference scale (0=no reside, 5=very strong residue). The formation of white stains was examined on the fabric Polo-Jersey (light blue, woven, 100% cotton). The formulations were sprayed on and the cloths were then washed with Persil washing powder at 40° C. and then dried. The process was repeated 4 times in order to be able to detect the formation of stains. The resulting stains were visually evaluated by trained experts (n =3) under a daylight lamp based on awarded score values (0=no stain, 6=very intense staining).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A deodorizing cosmetic agent in the form of a suspension, comprising:
1.0 to 35 wt % at least one hectorite,
55 to 95 wt % at least one oil that is liquid at 20° C. and 1,013 hPa,
less than 3.0 wt % water
wherein the deodorizing cosmetic agent does not include any aluminum-containing compounds.

2. The deodorizing cosmetic agent according to claim 1, wherein the agent includes a hectorite having a degree of purity above 95%.

3. The deodorizing cosmetic agent according to claim 1, wherein the agent includes a hectorite having a degree of purity above 97%.

4. The deodorizing cosmetic agent according to claim 1, wherein the agent includes a hectorite having a degree of purity above 99%.

5. The deodorizing cosmetic agent according claim 1, wherein the agent includes the hectorite, relative to the total weight of the agent, in an amount of 2.0 to 20%.

6. The deodorizing cosmetic agent according claim 1, wherein the agent includes the hectorite, relative to the total weight of the agent, in an amount of 3.0 to 15%.

7. The deodorizing cosmetic agent according to claim 1, wherein at least one oil is an oil having a vapor pressure of 0.01 kPa or more at 293.15 K.

8. The deodorizing cosmetic agent according to claim 1, wherein cyclopentasiloxane is included as an oil.

9. The deodorizing cosmetic agent according to claim 1, wherein 2-ethylhexyl palmitate is included as an oil.

10. The deodorizing cosmetic agent according to claim 1, wherein 2-isopropyl myristate is included as an oil.

11. The deodorizing cosmetic agent according to claim 1, wherein a mixture of cyclopentasiloxane, 2-ethylhexyl palmitate, and isopropyl myristate is included as the oil.

12. A non-therapeutic cosmetic method for preventing and/or reducing body odor, comprising:
applying the deodorizing cosmetic agent according to claim 1 to the skin and leaving the deodorizing cosmetic agent on the skin for a period of at least 1 hour.

13. The method of claim 12, wherein the skin is the skin of an armpit.

14. The method of claim 12, wherein the period of time is at least 2 hours.

15. The method of claim 12, wherein the period of time is at least 4 hours.

16. The method of claim 12, wherein the period of time is at least 6 hours
